(19) United States
(12) Patent Application Publication
(10) Pub. No.: US 2004/0029893 A1
(43) Pub. Date: Feb. 12, 2004

(54) Method of treatment of non-invasive mucositis

(76) Inventor: Edward M. Lane, Bridgeport, CT (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/624,609

(22) Filed: Jul. 23, 2003

Related U.S. Application Data
(60) Provisional application No. 60/398,207, filed on Jul. 23, 2002.
(60) Provisional application No. 60/401,972, filed on Aug. 7, 2002.
(60) Provisional application No. 60/407,182, filed on Aug. 29, 2002.

(51) Int. Cl. A61K 31/496; A61K 31/4196; A61K 31/4178

(52) U.S. Cl. 514/254.07; 514/383; 514/397

(57) ABSTRACT

This invention relates to a method of treating mucositis, and in particular fungal sinusitis in mammals, using oral medication, including azole antifungal agents such as, for example fluconazole and voriconazole, and with proton pump inhibitors such as esomeprazole.

This application claims the benefit of U.S. Provisional Application No. 60/398,207, filed Jul. 23, 2002, Provisional Application No. 60/401,972, filed Aug. 7, 2002, and Provisional Application No. 60/407,182, filed Aug. 29, 2002.

### BACKGROUND OF THE INVENTION

1. Technical Field

This invention relates to the field of medical science, and in particular to treatment of inflammatory conditions of the mucosa (mucositis), especially sinusitis and otitis media, and including acute maxillary sinusitis of fungal etiology, with orally administered drugs, such as azole antifungal drugs and proton pump inhibitors, including fluconazole, voriconazole and esomeprazole.

2. Description of the Background Art

Mucositis is an inflammation of the mucosa; sinusitis is a mucositis of the nasal and/or paranasal sinuses. The etiology of sinusitis, as of many other mucositis conditions, can be viral, bacterial, fungal, allergic, chemical, secondary to gastro-esophageal reflux disease (GERD) in the case of sinusitis, or due to combinations of several of these factors. The conditions may be acute, subacute, chronic or acute recurrent. A large segment of the population suffering from sinusitis or other mucositis (e.g., otitis media, mastoiditis, or vaginal, oral, colo-rectal and urinary tract inflammations), develop recurrences without any obvious cause.

Acute, subacute, acute recurrent, and chronic mucositis conditions such as sinusitis all traditionally have been treated with palliative therapy, such as decongestants and antihistamines, or with antibiotics such as penicillin, erythromycin, and tetracycline. Antibiotics such as amoxicillin and sulfa drugs may be used as first line treatment for sinus or other infections, but they also often are ineffective.

Recently, however, researchers at the Mayo Clinic have demonstrated that a high percentage of individuals suffering from recurrent sinusitis exhibit fungal growth in their sinuses. Recurrent sinusitis therefore in many cases may be related to fungal rather than bacterial infection, with bacterial super infection and/or allergic reactions to the fungus exacerbating the condition. If causative fungal infections can be eradicated, bacterial super infections could then be cleared naturally, without the use of antibiotics. The same reasoning applies to other mucositis conditions. Reducing the use of antibiotics is highly desirable, especially given both their lack of long-term efficacy in populations with recurrent mucositis and the unfortunate tendency to cause development of multi-drug resistant organisms.

Non-invasive, or allergic, fungal mucositis can be associated with Aspergillus, Biopolaris, Alternaria, Curvularia, Drechslera, Candida or Exserohilum species and often is recurrent and highly resistant to conventional treatments for infection. Aspergillus fumigatus and Biopolaris spicifera are common in some geographic areas. Unlike invasive fungal infections of the mucosa, in fungal mucositis the fungal hyphae are present in the mucin but do not invade soft tissue or bone. A non-surgical treatment for acute sinusitis or other mucositis or recurrent bouts of mucositis which eliminates the colonizing fungus and allows the tissues to return to their normal sterile state is not currently available.

The treatment of choice for non-invasive fungal sinusitis, once it has been diagnosed apart from bacterial sinusitis, has been surgery followed by oral and/or topical steroids. Systemic (oral) anti-fungal drugs have not been indicated in the absence of invasion of the tissues. Long-term follow-up is necessary in these patients, and a high degree of recurrence, often necessitating additional surgeries is seen. Even the most aggressive therapies often are ineffective in the long term. Blockage of the sinuses with fungal mucin often results in cycles of bacterial super infection, and the lack of proper drainage and aeration of the sinuses makes clearance of the fungal mucin very difficult. Allergic reactions resulting in inflammation can worsen the condition and make relapses more likely. This phenomenon is seen in otitis media when the Eustachian tube becomes blocked.

Surgical treatment carries its own risks, for example scarring which can produce blockage of the area, exacerbating drainage problems. Removal of both necrotic and healthy tissue often is necessary because failure to remove all polyps and mucin plugs almost guarantees recurrence of the condition. A second or subsequent surgery therefore is more dangerous because less anatomy is present directly below the brain. A treatment method for fungal sinusitis and other mucositis that avoids these dangers and this invasive and uncomfortable treatment is needed in the art.

Complete removal of the fungus is necessary to avoid a continuing inflammatory reaction to fungal allergens, however complete removal usually is not possible with current surgical techniques, depending on the location of fungus, and patients are constantly re-exposed to the same or different inhaled fungi from the environment. New or continued exposure to fungal allergens results in inflammation and production of mucous, which results in partial or complete blockage of the sinus or other area, which in turn exacerbates the exposure to allergens and provides a location for fungal growth. Even using surgery with long-term follow-up treatments directed toward avoiding immune reaction, inflammation and blockage, this vicious cycle is difficult to break.

It is important in most patients to allow the mucus in the sinuses to drain adequately. This has been accomplished with nasal sprays containing small amounts of cortisone to reduce the inflammation inside the nose and around the ostia (the opening from the sinuses into the nose) and Eustachian tube, for example. Nasal lavage with solutions containing steroids to reduce inflammation and regular monitoring for recurring obstruction after surgery are helpful, but difficult, expensive and unpleasant, and sometimes involve undesirable side-effects. Decongestants can be employed to reduce the swelling inside the nose, and drugs that reduce the thickness of the mucus have been employed. Home remedies, including eucalyptus, garlic and steam also have been suggested. Currently, the disease is considered chronic and potentially life-long.

Cessation of the oral corticosteroid treatment, even in a tapered manner, may result in rapid recurrence of symptoms. Many patients have recurrent sinusitis and must be subjected to multiple surgeries and long courses of steroid drugs. Some patients may take corticosteroid medication for a year or longer. Aggressive allergy management therapy also is used to manage fungal sinusitis and other mucositis. Treatments such as antiinflammatory nasal sprays, saline lavage antihistamines decongestants and immunotherapy have been used, alone or in combination with surgery and/or steroid therapy. Ear drops, vaginal douches, enemas, suppositories and other therapies may be used to apply such medications to the desired area, as is known in the art. These treatments sometimes alleviate symptoms, but do nothing to eliminate the root cause of the problem. These factors make a non-surgical, simple treatment for fungal mucositis very desirable, particularly a treatment which sterilizes fungal infected areas and promotes drainage and clearance of any bacterial super infection that may be present.

In U.S. Pat. No. 6,291,500, the specification indicates that azole antifungal agents, including fluconazole and voriconazole, can be administered to place them in contact with causative fungal organisms within the nasal-paranasal mucus and, thereby, treat or prevent chronic rhino-sinusitis symptoms. While Ponikau mentions the possibility of oral administration to effect mucoadministration to the gastrointestinal tract or for indirect mucoadministration to nasal-paranasal mucosa provided that the administered agent comes into physical contact with the nasal-paranasal mucus, the patent disclosures emphasize a minimal inhibitory concentration on the mucosal surface that is necessary for effective treatment. According to the patent's disclosures, administration should involve direct application to the mucus at a MIC (minimum inhibitory concentration) of 0.25 μg/mL to greater than about 64 μg/mL.

Orally active azole antifungal drugs have been described. See U.S. Pat. No. 4,404,216. These drugs have been used effectively for invasive fungal infections due to Candida, Aspergillis, and other fungi. Azole antifungal agents such as fluconazole and voriconazole exert their effect by inhibiting cytochrome p450 14a-desmethylase (P45014DM), an enzyme in the steroid biosynthesis pathway. Voriconazole has in vitro antifungal activity against a number of species and is considered to be effective in vivo against Candida spp. and Cryptococcus neoformans as well as Aspergillis spp., including fluconazole-resistant Candida species such as C. krusei and C. guilliermondii. Fluconazole (Diflucan®), itraconazole (Sporanox®) and voriconazole (Vfend®) have been approved by the FDA for various types of invasive fungal infections. These drugs are synthetic triazole antifungal agents, available as tablets for oral administration. Prescribing information for these drugs list the following indications for usage. Fluconazole: vaginal candidiasis; oropharyngeal and esophageal candidiasis; Candida urinary tract infections, peritonitis, and systemic Candida infections including candidemia, disseminated candidiasis, and pneumoma; and cyptococcal meningitis. Voriconazole: invasive aspergillosis and serious fungal infections caused by Scedosporium apiospermum and Fusarium spp. Itraconazole: blastomycosis, histoplasmosis and aspergillosis in immunocompromised patients and onychomycosis in non-immunocompromised patients. Fluconazole also has been used to decrease the incidence of candidiasis in patients undergoing bone marrow transplantation who receive cytotoxic chemotherapy and/or radiation therapy.

Esomeprazole, available as Nexium®, is a proton pump inhibitor (PPI) that suppresses the final step in gastric acid production. Esomeprazole is an S-isomer of omeprazole. It acts by suppressing gastric acid secretion by specific inhibition of the H⁺/K⁺-ATPase in the gastric parietal cells. Esomeprazole is indicated for the treatment of erosive esophagitis associated with gastro-esophageal reflux disease (GERD). GERD is virtually universal. In most people it remains physiologic. When the frequency or duration of GERD becomes severe enough to induce symptoms or histologic changes of chronic inflammation, GERD becomes pathologic. GERD has been shown to play an important role in acute and chronic inflammatory disorders of the airway. In addition, there is a growing body of evidence that GERD plays a significant role in the development of a number of diseases and symptom complexes, including cancer of the larynx, laryngeal and tracheal stenosis, reflux, laryngitis, globus, chronic cough, otitis media, and sinusitis. See, for example, Levinson et al., “Sinusitis in Children—Diagnosis and Treatment,” Med. J. Allina, vol. 5, no. 1, Winter 1995. Other proton pump inhibitors which may be used in methods of this invention include but are not limited to omeprazole (Prilosec®), lansoprazole (Prevacid®), pantoprazole (Protonix®) and others.

The published literature has reported that systemic antifungal drugs are expected to be ineffective for treatment of non-invasive fungal conditions because the fungi are located outside of the range of drug circulation. The conventional wisdom in the art indicates that oral antifungal treatment should not be effective because allergic-type fungal mucositis is not tissue-invasive. The fungus therefore would remain out of reach of the bloodstream and therefore the antifungal agent. See, e.g., Schubert, Ann. Allergy Asthma Immunol. 85(2):90-97, 2000. To be effective, the literature teaches that the drug must be secreted in the mucous, which has not been shown to occur. Therefore only local or topical mucoadministration, by spray, irrigation, packing with gauze impregnated with the medication, etc., has been considered to be useful for treatment of noninvasive mucositis conditions. Topical application of antifungal medication to the entire affected area, however, is problematic, especially when the area, such as a nasal or paranasal sinus, is completely blocked and when all or part of the affected area is anatomically inaccessible. The goal in using these topical antifungals has been to maintain a reduced level of fungal organisms in the effected tissues to prevent or reduce symptoms.

An improved treatment for non-invasive, allergic-type sinusitis would be highly desirable; in particular, a treatment that is safe, relatively free of side effects, does not require surgical intervention, and effectively sterilizes all of the affected mucosal tissues is needed in the art. Ideally, a treatment would be safe enough to repeat should the condition recur due to new exposure to fungus through the environment, even multiple times. Currently, no such treatment is available on the market. The goal of current therapy has been to minimize the symptomology of the disease and delay the need for recurrent surgery. Therefore, a method to treat fungal mucositis to affect a cure rather than merely to minimize its impact on the patient would be highly desirable and serves a great need in the art. Consequently, the inventive treatments disclosed here represent a useful advance in the art of medical treatment of these conditions.

### SUMMARY OF THE INVENTION

An objective of certain embodiments of this invention is to provide a treatment for mucositis in a patient without the need for surgical intervention, using oral medication.

Accordingly, embodiments of this invention provide a method of treating mucositis in a patient in need thereof without surgery, which comprises orally administering to said patient a therapeutically effective amount of an oral medication, for example a proton pump inhibitor and/or azole antifungal drug. In preferred methods, the azole antifungal drug is fluconazole or voriconazole and the proton pump inhibitor is Esomeprazole. Treatment usually is given for at least about 10 days up to about 30 days, or longer. Preferred methods comprise a treatment for mucositis of non-invasive fungal etiology, in particular fungal otitis media, nasal sinusitis and paranasal sinusitis.

A number of preferred aspects of the invention will be described below.

### DETAILED DESCRIPTION OF THE INVENTION

The invention relates to methods of treating mucositis, particularly non-invasive fungal mucositis (allergic-type) without the need for surgery, using oral medication. Embodiments of the present invention are effective to treat mucositis of a fungal etiology, including non-invasive fungal colonization of the mucosa of the nasal and paranasal sinuses, and the inner and middle ear. Additional anatomical sites where these types of conditions may arise include the mastoid, vagina, mouth and the colon and rectum. Certain preferred treatments of the invention have particular importance in the treatment of acute maxillary sinusitis by orally administering an azole antifungal drug such as fluconazole or voriconazole, and optionally also a proton pump inhibitor such as esomeprazole.

Treatment of sinusitis may involve oral administration of esomeprazole omeprazole, lansoprazole, pantoprazole or any proton pump inhibitor (alone or in conjunction with an oral antifungal drug) to break a link in the cycle of chronic sinusitis by eliminating a constant source of irritation to the mucous membranes which may allow inflammation and colonization by organisms to proceed. Sinusitis in GERD patients is thought to be caused by the low pH of the reflux gastric fluid causing inflammation and edema at the sinus ostia. The sinus osteal inflammation then progresses to obstruction of the ostia, thus allowing bacteria or other organisms to proliferate within the sinuses, and an infection to occur. The sinus infection therefore is secondary to inflammation at the sinus ostia, caused by the acid reflux. In patients with sinusitis who also suffer from gastro-esophageal reflux disease (GERD), esomeprazole may be used as a sole oral therapy or may be used in conjunction with oral antifungal drugs such as fluconazole or voriconazole. Antifungal oral medication may be used concurrently or sequentially with proton pump inhibitor treatment but preferably is used concurrently.

Treatment of sinusitis with esomeprazole is particularly important in patients where the frequency or duration of GERD becomes severe enough to induce symptoms or histologic changes of chronic inflammation. In these cases, GERD must be treated to effect a cure for the sinusitis. The treatment provided by the invention for sinusitis in GERD patients treats the low pH of the reflux gastric fluid and thereby diminishes the causation of inflammation and edema at the sinus ostia. The treatment of these embodiments of the invention is particularly effective for patients with acute maxillary sinusitis, by inhibiting acid reflux, allowing the inflammation at the sinus ostia to be resolved and the sinuses to drain spontaneously. If a sinusitis patient on sole oral esomeprazole therapy does not improve or worsens clinically, oral antifungal agent therapy according to the invention may be added or substituted.

Generally, effective amounts of a proton pump inhibitor raise the pH of the gastric contents above a pH of about 4.0, preferably above a pH of about 4.5, or most preferably above a pH of about 5.0, particularly in acute cases. Traditionally, proton pump inhibitors are administered with the goal of raising the pH of gastric fluid above 4.0, but a higher dosage is preferred for the treatment of sinusitis, to raise the pH above 5.0, which is thought to be necessary to control the extra-esophageal symptoms of GERD. A pH probe (e.g., for 24 hours) measuring the pH in the distal esophagus and the hypopharnyx can be recorded prior to the initiation of the therapy to ensure that only patients with GERD are treated for sinusitis according to these embodiments of the invention, using esomeprazole. To demonstrate that the rise in pH was sustained, the 24 hour pH probe can be repeated at the completion of therapy.

Proton pump inhibitor drugs are well known in the art, as are methods for measuring the pH of gastric contents. Therefore an effective amount can be determined easily by any person of skill in the art. In general, effective doses of proton pump inhibitors such as esomeprazole are about 1 to about 50 mg/kg/day given at intervals of from one to two times a day, however it is considered routine by those of skill in the art to adjust doses based on factors individual to each patient. Therefore, doses outside these ranges are contemplated. Treatment for sinusitis using a proton pump inhibitor preferably proceeds for at least 10 days. Preferably, the drug is administered for about 10 days to about 30 days, however treatment may be continued for longer periods and may continue indefinitely at the original dose or a lower, maintenance dose to prevent reinjury of nasal, paranasal or other mucosa due to GERD. This treatment therefore is contemplated for both chronic and acute sinusitis. Acute maxillary sinusitis in the presence of GERD is most advantageously treated by these embodiments of the invention.

The treatment of the invention can include auxiliary and supplemental treatments as determined to be useful or necessary by a physician and can include direct mucosal administration of medications, such as by infusion or irrigation, injection or any method known in the art. In addition, other concurrent or sequential therapies can be employed according to currently recommended regimens or in amounts or for times reduced from current practice. For example, systemic steroids, intranasal steroids, intranasal lavage with saline (normotonic or hypertonic) and/or topical antifungal medication (e.g., Nystatin powder). Oral antifungal medication, such as fluconazole or voriconazole at dosages and treatment regimens as described herein for azole antifungal medications may be used concurrently or sequentially with esomeprazole treatment.

Both fluconazole and voriconazole, as well as other azole antifungal drugs such as itraconazole are per se known. As is known in the art, these drugs have different activity profiles. Therefore, if fungal culture or any other method for identifying the causative fungal species for mucositis in an individual patient is available, the treating physician can chose an antifungal medication which would be preferred for an individual patient. These types of decisions are considered routine to those in the art of medicine. Fluconazole may be used preferably against mucositis caused by Candida spp. Voriconazole is preferred against Aspergillis spp., Candida spp., Scedosporium spp., Fusarium spp., Histoplasma capsulatum, Blastomyces dermatitidis and Cryptococeus neoformans, for example. However, it is well within the ability of the clinician to chose a preferred azole antifungal medication for an individual patient and to adjust an appropriate dose.

The causative organism of mucositis, such as sinusitis, cannot always be identified by culture or other methods. The presence of fungus is common, however, as the primary or at least a secondary cause of symptoms, therefore the methods of the invention also are suitable for treatment of any acute and especially any chronic or recurrent mucositis, even when a definitive cause has not been determined. This is possible because of the relatively non-invasive, simple and inexpensive nature of these treatments.

The treatments of the invention employ reasonable doses of oral medication over reasonable periods of time, making the treatments convenient for the patients. A typical dose of azole antifungal drug is 200 milligrams per day (more broadly, from 50 to 500 milligrams) for a normal adult male over a treatment period of from about 10 days to about 30 days. Shorter treatment periods are contemplated but must be done with further study or daily monitoring of the patient. Longer treatment periods are also contemplated, but should be done after full evaluation of a patient's progress.

For treatment of allergic-type non-invasive fungal sinusitis, oral fluconazole may be administered at a dose of about 10 mg/day to about 1000 mg/day, which may be divided into 1-4 individual doses per day, for a period of about 10 days to about 4-6 weeks. It is advantageous to continue treatment at least about 14 days following resolution of symptoms. Preferred treatment regimens include doses of about 50 mg to about 400 mg/day or most preferably about 100 mg to about 200 mg/day, and are continued for at least 10 days. The length of treatment may be 1 day to about 30 days and preferably about 10 to about 30 days or more. The daily dosages above may be divided into subdoses or given once daily.

Dosages of voriconazole for oral treatment of non-invasive fungal sinusitis generally are about 100 mg/day to about 1200 mg/day for a period of about 10 days to about 4 weeks, or at least about 14 days following resolution of symptoms. Preferably, voriconazole is given at doses of about 100 mg to about 600 mg/day or most preferably about 400-600 mg/day, and is continued for at least about 10 days. The length of treatment may be 1 day to about 30 days and preferably about 10 to about 30 days or more. The preferred dose of about 400-600 mg/day corresponds roughly to about 3-4 mg/kg every 12 hours. The daily dosages above may be divided into 2-4 subdoses or given once daily.

Esomeprazole treatment of non-invasive fungal sinusitis involves administration of the drug orally at a dose of about 0.1 mg/kg/day to about 50 mg/kg/day, for a period of about 2 to about 6 weeks. It is advantageous to continue treatment at least about 14 days following resolution of symptoms. Preferably the drug is given at a dose of about 10 mg once a day to about 80 mg/twice a day, and is continued for at least 14 days. Normal dosages are usually about 40 mg, once a day, or about 1-5 mg/kg/day. The length of treatment may be 10 days to about 30 days and preferably about 4 weeks or more, including treatment for up to a year or longer. In all cases, the invention entails orally administering an effective dose of drug at intervals and for a treatment period effective to lessen or cure the patient of the mucositis. Dosages and treatment regimens as described above also are suitable for non-invasive fungal mucositis in different anatomical locations. The term “therapeutically effective amount” as used herein refers to an amount sufficient to ameliorate or cure a mucositis condition. Clinicians are accustomed to judge sufficiency of such treatments and may increase or decrease the ranges given above as guidelines according to their clinical judgement. Therefore, dosages and treatment lengths outside these ranges are contemplated as part of this invention.

1. A method of treating mucositis in a patient in need thereof without surgery, which comprises orally administering to said patient a therapeutically effective amount of an oral medication selected from the group consisting of an azole antifungal agent, a proton pump inhibitor and a combination thereof.

2. A method of claim 1 which comprises orally administering to said patient a therapeutically effective amount of an azole antifungal agent.

3. A method of claim 1 which comprises orally administering to said patient a therapeutically effective amount of a proton pump inhibitor.

4. A method of claim 1 which comprises orally administering to said patient a therapeutically effective amount of an azole antifungal agent and a therapeutically effective amount of a proton pump inhibitor.

5. A method of claim 1 wherein said azole antifungal agent is selected from the group consisting of fluconazole, voriconazole and itraconazole.

6. A method of claim 1 wherein said azole antifungal agent is administered in an amount of about 10 to about 1200 mg/day.

7. A method of claim 1 wherein said azole antifungal agent is administered in an amount of about 50 to about 600 mg/day.

8. A method of claim 1 wherein said azole antifungal agent is administered in an amount of about 200 mg/day to about 400 mg/day.

9. A method of claim 1 wherein said azole antifungal agent is administered for at least 10 days.

10. A method of claim 1 wherein said azole antifungal agent is administered for about 10 days to about 30 days.

11. A method of claim 1 wherein said mucositis is non-invasive fungal mucositis.

12. A method of claim 1 wherein said non-invasive fungal mucositis is otitis media.

13. A method of claim 1 wherein said non-invasive fungal mucositis is nasal and/or paranasal sinusitis.

14. A method of claim 1 wherein said proton pump inhibitor is esomeprazole.

15. A method of claim 1 wherein said proton pump inhibitor is administered in an amount that raises the pH of gastric fluid above about pH 4.0.

16. A method of claim 1 wherein said proton pump inhibitor is administered in an amount that raises the pH of gastric fluid above about pH 5.0.
